(19) United States
(12) Patent Application Publication
(10) Pub. No.: US 2025/0195137 A1
(43) Pub. Date: Jun. 19, 2025

(54) OPEN-TIPPED RADIOFREQUENCY PERFORATION DEVICE

(71) Applicant: Boston Scientific Medical Device Limited, Galway (IE)

(72) Inventors: Sherif Ramadan, Hamilton (CA); Gareth Davies, Toronto (CA); Lauren Koon, Mississauga (CA); Matthew Choi, Markham (CA)

(21) Appl. No.: 18/984,847

(22) Filed: Dec. 17, 2024

Related U.S. Application Data
(60) Provisional application No. 63/611,432, filed on Dec. 18, 2023.

(51) Int. Cl. A61B 18/14; A61B 18/00

(52) U.S. Cl. CPC A61B 18/1492; A61B 2018/00357; A61B 2018/1405

(57) ABSTRACT

A radiofrequency perforation device for perforating a tissue is disclosed. The radiofrequency perforation device includes an elongate member defining a lumen and extending from a proximal portion including a hub to a distal portion, the distal portion including an open port housing having at least one aperture and an open port outer diameter. The radiofrequency perforation device also includes a functional distal tip located at the distal portion of the elongate member having a distal face and including a distal tip electrode and a distal tip outer diameter. The distal portion further includes a tapered arm connecting the open port housing to the functional distal tip, wherein the open port housing is proximal to the functional distal tip and wherein the functional distal tip outer diameter is less than the open port housing outer diameter. The distal face is a half-dome shape such that it reduces coring of the tissue.

### CROSS REFERENCE TO RELATED APPLICATIONS

The present application claims priority to, and the benefit of, U.S. provisional patent application No. 63/611,432, filed Dec. 18, 2023, the entire disclosure of which is incorporated herein by reference.

### TECHNICAL FIELD

The present invention relates generally to methods and devices usable to deliver energy within the body of a patient. More specifically, the present invention is concerned with a radiofrequency perforation apparatus.

### BACKGROUND

Devices currently exist for creating a puncture, channel, or perforation within a tissue located in a body of a patient. One such device is the Brockenbrough Needle, which is commonly used to puncture the atrial septum of the heart. This device is a stiff elongated needle, which is structured such that it may be introduced into a body of the patient via the femoral vein and directed towards the heart. This device relies on the use of mechanical force to drive the sharp tip through the septum.

Alternatively, devices currently exist for access to the epicardial space. Access to the space may be initiated with a mechanical puncture device using a large bore needle, for example a Tuohy-style needle. These needles for access to the epicardial space are associated with high clinical complication rates. Additionally, the design of epicardial access needles typically include side ports and lack a forward-facing lumen aperture. Devices having a forward-facing aperture are typically better in facilitating the use of a guidewire than a side port device.

Furthermore, in radiofrequency (RF) ablation procedures when utilizing an NRG® needle two issues currently exist. First, the NRG® needle does not allow for advancement of a 0.014″ guidewire after puncture which disrupts workflow. The 0.014″ guidewire is typically used to insert the guidewire and device into the left superior pulmonary vein to maintain access into the heart and locate anatomical features which assists in advancing sheaths for subsequent procedures. Second, currently, the NRG® needle contains side ports for contrast injection and pressure readings. A more accurate placement of these markers would be at the distal end of the device which may enable smoother workflow and more precise tracking of the tip of the needle's location throughout a procedure. However, creating an open tip at the distal end of the device may lead to tissue coring when RF energy is applied.

Against this background, there exists a continuing need in the industry to provide improved radiofrequency perforation devices and methods to gain access to the epicardial and transseptal space. An object of the present invention is therefore to provide such a radiofrequency perforation apparatus.

### SUMMARY

In Example 1, a radiofrequency perforation device for perforating a tissue includes an elongate member defining a lumen and extending from a proximal portion including a hub to a distal portion, the distal portion including an open port housing having at least one aperture and an open port outer diameter. The radiofrequency perforation device also includes a functional distal tip located at the distal portion of the elongate member having a distal face and including a distal tip electrode and a distal tip outer diameter; wherein the distal portion further includes a tapered arm connecting the open port housing to the functional distal tip, wherein the open port housing is proximal to the functional distal tip and wherein the functional distal tip outer diameter is less than the open port housing outer diameter; and wherein the distal face is a half-dome shape such that it reduces coring of the tissue.

Example 2 is the radiofrequency perforation device of Example 1 wherein the open port housing further includes a proximal edge that surrounds the aperture of the open port housing.

Example 3 is the radiofrequency perforation device of any of Examples 1-2 wherein the tapered arm is formed from a section of the proximal edge.

Example 4 is the radiofrequency perforation device of Example 1 wherein the functional distal tip protrudes next to and overshadows the aperture without obstructing the aperture.

Example 5 is the radiofrequency perforation device of Example 1 wherein the distal face includes any geometric shape with a curved segment having no sharp edges.

Example 6 is the radiofrequency perforation device of Example 5 wherein the curve segment of the distal face allows for the safe advancement of the functional distal tip through a perforation created by the distal tip electrode.

Example 7 is the radiofrequency perforation device of Example 1 wherein the aperture is a forward-facing open port, and wherein the forward-facing open port facilitates the insertion of a guidewire from the proximal portion of the elongate member through to the forward-facing open port.

Example 8 is the radiofrequency perforation device of Example 8 wherein the guidewire is a 0.014″ guidewire.

Example 9 is the radiofrequency perforation device of any of Examples 1-7 wherein the functional distal tip is curved towards the open port to overshadow the open port without physically obstructing the open port.

Example 10 is the radiofrequency perforation device of Example 10 wherein the curved angle of the functional distal tip is between 35° and 55°.

Example 11 is the radiofrequency perforation device of any of Examples 1-7 wherein the radiofrequency perforation device further includes a side port that is proximal to the open port.

Example 12 is the radiofrequency perforation device of Example 1 wherein the functional distal tip includes two or more distal tip radiofrequency electrodes.

Example 13 is the radiofrequency perforation device of Example 1 wherein the distal portion further includes an inner section defining the inner portion of the functional distal tip, including the distal tip electrode, and the inner portion of the arm, and wherein the inner section is made of any biocompatible electrically conductive material and capable of transferring radiofrequency energy supplied by an external RF generator to the distal tip electrode and subsequent delivery to a target tissue.

Example 14 is the radiofrequency perforation device of any of Examples 1-14 wherein the distal portion further includes an outer section defining the outer portion of the functional distal tip, including the curved segment of the functional distal tip, and the outer portion of the arm, and wherein the outer section is made of any electrically insulative material.

Example 15 is the radiofrequency perforation device of Example 1 wherein a fluid exits the aperture, and wherein the fluid exits the aperture at an angle to the longitudinal.

In Example 16, a radiofrequency perforation device for perforating a tissue includes an elongate member defining a lumen and extending from a proximal portion including a hub to a distal portion, the distal portion including an open port housing having at least one aperture and an open port outer diameter. The radiofrequency perforation device further includes functional distal tip located at the distal portion of the elongate member having a distal face and including a distal tip electrode and a distal tip outer diameter; wherein the distal portion further includes a tapered arm connecting the open port housing to the functional distal tip, wherein the open port housing is proximal to the functional distal tip and wherein the functional distal tip outer diameter is less than the open port housing outer diameter; and wherein the distal face is a half-dome shape such that it reduces coring of the tissue.

Example 17 is the radiofrequency perforation device of Example 16 wherein the open port housing further includes a proximal edge that surrounds the aperture of the open port housing.

Example 18 is the radiofrequency perforation device of Example 17 wherein the tapered arm is formed from a section of the proximal edge.

Example 19 is the radiofrequency perforation device of Example 16 wherein the functional distal tip protrudes next to and overshadows the aperture without obstructing the aperture.

Example 20 is the radiofrequency perforation device of Example 16 wherein the distal face includes any geometric shape with a curved segment having no sharp edges.

Example 21 is the radiofrequency perforation device of Example 16 wherein the aperture is a forward-facing open port, and wherein the forward-facing open port facilitates the insertion of a guidewire from the proximal portion of the elongate member through to the forward-facing open port.

Example 22 is the radiofrequency perforation device of Example 21 wherein the functional distal tip is curved towards the open port to overshadow the open port without physically obstructing the open port.

Example 23 is the radiofrequency perforation device of Example 23 wherein the curved angle of the functional distal tip is between 35° and 55°.

Example 24 is the radiofrequency perforation device of Example 21 wherein the radiofrequency perforation device further includes a side port that is proximal to the open port.

Example 25 is the radiofrequency perforation device of Example 16 wherein the functional distal tip includes two or more distal tip RF electrodes.

Example 26 is the radiofrequency perforation device of Example 16 wherein a fluid exits the aperture, and wherein the fluid exits the aperture at an angle to the longitudinal.

In Example 27, an epicardial or transseptal crossing system for perforating a tissue includes a dilator having a dilator body defining a dilator lumen and a tapered distal tip. The crossing system further includes an elongate member defining a lumen and extending from a proximal portion including a hub to a distal portion, the distal portion including an open port housing having at least one aperture and an open port outer diameter. The crossing system also includes a functional distal tip located at the distal portion of the elongate member having a distal face and including a distal tip electrode and a distal tip outer diameter; wherein the distal portion further includes a tapered arm connecting the open port housing to the functional distal tip, wherein the open port housing is proximal to the functional distal tip and wherein the functional distal tip outer diameter is less than the open port housing outer diameter; and wherein the distal face is a half-dome shape with no sharp edges, such that it reduces coring of the tissue.

Example 28 is the crossing system of Example 27 wherein the open port housing further includes a proximal edge that surrounds the aperture of the open port housing.

Example 29 is the crossing system of Example 28 wherein the tapered arm is formed from a section of the proximal edge.

Example 30 is the crossing system of Example 27 wherein the functional distal tip protrudes next to and overshadows the aperture without obstructing the aperture.

Example 31 is the crossing system of Example 27 wherein the aperture is a forward-facing open port.

Example 32 is the crossing system of Example 31 wherein the functional distal tip is curved towards the open port to overshadow the open port without physically obstructing the open port.

Example 33 is the crossing system of Example 27 wherein the distal portion further includes an inner section defining the inner portion of the functional distal tip, including the distal tip electrode, and the inner portion of the arm, and wherein the inner section is made of any biocompatible electrically conductive material and capable of transferring radiofrequency energy supplied by an external RF generator to the distal tip electrode and subsequent delivery to a target tissue.

Example 34 is the crossing system of Example 33 wherein the distal portion further includes an outer section defining the outer portion of the functional distal tip, including the curved segment of the functional distal tip, and the outer portion of the arm, and wherein the outer section is made of any electrically insulative material.

In Example 35, a method of epicardial or transseptal crossing includes providing an elongate member defining a lumen and extending from a proximal portion including a hub to a distal portion, the distal portion including an open port housing having at least one aperture and an open port outer diameter. The method further includes advancing a functional distal tip located at the distal portion of the elongate member having a distal face and including a distal tip electrode and a distal tip outer diameter; wherein the distal portion further includes a tapered arm connecting the open port housing to the functional distal tip, wherein the open port housing is proximal to the functional distal tip and wherein the functional distal tip outer diameter is less than the open port housing outer diameter; and wherein the distal face is a half-dome shape such that it reduces coring of the tissue.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIGS. 1A-1D are schematic illustrations of a medical procedure within a patient's heart for gaining access to the transseptal and epicardial space, according to embodiments of the present disclosure.

FIG. 2 is a schematic illustration of a dilator and radiofrequency perforation device of the transseptal access system illustrated in FIGS. 1A-1D, according to embodiments of the present disclosure.

FIGS. 3A-3D are cross-sectional and schematic illustrations of a distal portion of a radiofrequency perforation device, as illustrated in FIG. 2, with an open port and a functional distal tip, according to embodiments of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

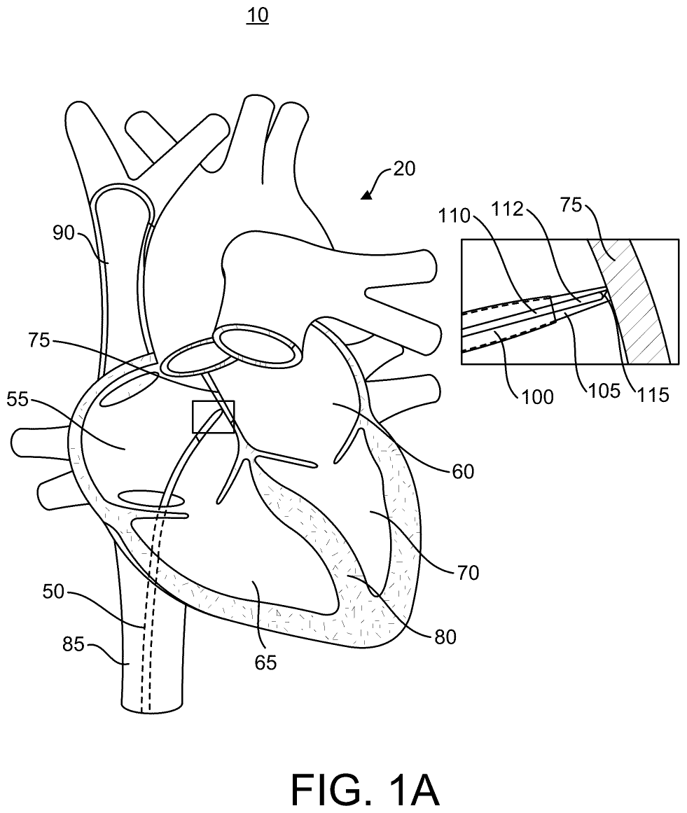

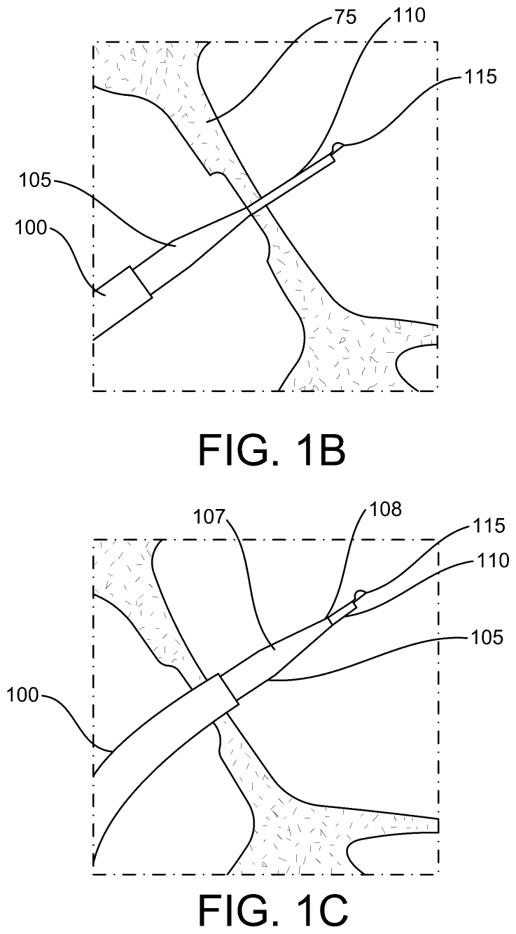

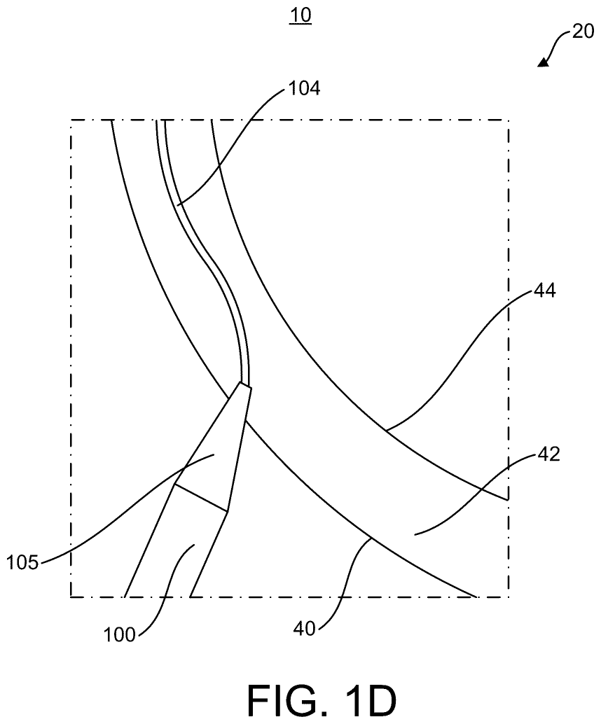

FIGS. 1A-1D are schematic illustrations of a medical procedure within a patient's heart for gaining access to the transseptal and epicardial space, according to embodiments of the present disclosure. FIGS. 1A-1C are illustrations of a medical procedure 10 within a patient's heart 20 utilizing a transseptal access system 50. As is known, the human heart 20 has four chambers, a right atrium 55, a left atrium 60, a right ventricle 65 and a left ventricle 70. Separating the right atrium 55 and the left atrium 60 is an atrial septum 75 and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

The medical procedure 10 illustrated in FIGS. 1A-1C is an exemplary embodiment for providing access to the left atrium 60 using the transseptal access system 50 for subsequent deployment of the aforementioned diagnostic and/or therapeutic devices within the left atrium 60. As shown in FIGS. 1A-1C, target tissue site can be defined by tissue on the atrial septum 75. In the illustrated embodiment, the target site is accessed via the IVC 85, for example through the femoral vein, according to conventional catheterization techniques. In other embodiments, access to the target site on the atrial septum 75 may be accomplished using a superior approach wherein the transseptal access system 50 is advanced into the right atrium 55 via the SVC 90.

In the illustrated embodiment, the transseptal access system 50 includes an introducer sheath 100, a dilator 105 having a dilator body 107 and a tapered distal tip portion 108, and a radiofrequency (RF) perforation device 110 having a distal end portion 112 terminating in a tip electrode 115. As shown, in the assembled use state illustrated in FIGS. 1A-1C, the RF perforation device 110 can be disposed within the dilator 105, which itself can be disposed within the sheath 100. In one embodiment in which the transseptal access system 50 is deployed into the right atrium 55 via the IVC 85, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator 105 may then be introduced into the sheath 100 and over the guidewire, and advanced through the sheath 100 into the SVC 90. Alternatively, the dilator 105 may be fully inserted into the sheath 100 prior to entering the body, and both may be advanced simultaneously towards the heart 20. When the guidewire, sheath 100 and dilator 105 have been positioned in the SVC 90, the guidewire is removed from the body, and the sheath 100 and the dilator 105 are retracted so that their distal ends are positioned in the right atrium 55. The RF perforation device 110 described can then be introduced into the dilator 105, and advanced toward the heart 20. In certain embodiments, the dilator may be introduced into the body without a need for the sheath 100.

Subsequently, the user may position the distal end of the dilator 105 against the atrial septum 75, which can be done under imaging guidance. The RF perforation device 110 is then positioned such that the tip electrode 115 is aligned with or protruding slightly from the distal end of the dilator 105. The dilator 105 and the RF perforation device 110 may be dragged along the atrial septum 75 and positioned, for example against the fossa ovalis of the atrial septum 75 under imaging guidance. A variety of additional steps may be performed, such as measuring one or more properties of the target site, for example an electrogram or ECG (electrocardiogram) tracing and/or a pressure measurement, or delivering material to the target site, for example delivering a contrast agent. Such steps may facilitate the localization of the tip electrode 115 at the desired target site. In addition, tactile feedback provided by medical RF perforation device 110 is usable to facilitate positioning of the tip electrode 115 at the desired target site.

With the tip electrode 115 and dilator 105 positioned at the target site, energy is delivered from an energy source, e.g., an RF generator, through the RF perforation device 110 to the tip electrode 115 and the target site. In some embodiments, the energy is delivered at a power of at least about 5 W at a voltage of at least about 75 V (peak-to-peak), and functions to vaporize cells in the vicinity of the tip electrode 115, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 1 second and about 5 seconds.

With the tip electrode 115 of the RF perforation device 110 having crossed the atrial septum 75, the dilator 105 can be advanced forward, with the tapered distal tip portion 108 operating to gradually enlarge the perforation to permit advancement of the distal end of the sheath 100 into the left atrium 60.

In some embodiments, the distal end portion 112 of the RF perforation device 110 may be pre-formed to assume an atraumatic shape such as a J-shape, a pigtail shape or other shape selected to direct the tip electrode 115 away from the endocardial surfaces of the left atrium 60. Examples of such RF perforation devices can be found, for example, in U.S. patents application Ser. Nos. 16/445,790 and 16/346,404 assigned to Baylis Medical Company, Inc. The aforementioned pre-formed shapes can advantageously function to minimize the risk of unintended contact between the tip electrode 115 and tissue within the left atrium 60 and can also operate to anchor the distal end portion 112 within the left atrium 60 during subsequent procedural steps. For example, in some embodiments, the RF perforation device 110 can be structurally configured to function as a delivery rail for deployment of a relatively larger bore therapy delivery sheath and associated dilator(s). In such embodiments, the dilator 105 and the sheath 100 are withdrawn following deployment of the distal end portion 112 of the RF perforation device 110 into the left atrium 60. The anchoring function of the pre-formed distal end portion 112 inhibits unintended retraction of the distal end portion 112, and corresponding loss of access to the perforated site on the atrial septum 75, during such withdrawal.

As shown in FIG. 1D, still another medical procedure 10 developed for diagnosing or treating physiological ailments originating within the heart 20 includes epicardial ablation to help restore a regular heart rhythm. As illustrated, the heart includes a pericardium 40, a pericardial cavity 42 and a myocardium 44. The heart 20 is typically approached using a subxiphoid approach. Epicardial access is achieved via puncturing a layer of the pericardium 40 while avoiding the myocardium 44 of the heart. The pericardium 40 is a tough, double-walled, fibroelastic sac encompassing the heart 20 and the roots of the great vessels. The pericardium 40 includes two layers, an outer layer made of strong connective tissue often referred to as the fibrous pericardium, and an inner layer made of serous membrane often referred to as the serous pericardium. The mesothelium, or mesothelial cells, that constitutes the serous pericardium also covers the myocardium of the heart as epicardium, resulting in a continuous serous membrane invaginated onto itself as two opposing surfaces such as over the fibrous pericardium 40 and over the heart 20. This creates a pouch-like virtual or potential space around the heart enclosed between the two opposing serosal surfaces, often referred to as the pericardial space or pericardial cavity 42.

In some embodiments, the pericardium 40 may be punctured with a needle. Once punctured, a dilator 105 is advanced to dilate the puncture created by the needle through the pericardium 40. In some embodiments, a sheath 100 may be advanced with the dilator 105. In other embodiments, the sheath 100 may be advanced afterwards. The sheath 100 and the dilator 105 may then be withdrawn to leave the guidewire 104 in the pericardial cavity 42. Minimally invasive access to the epicardium is required for diagnosis and treatment of a variety of arrhythmias and other conditions. During epicardial ablation, tiny scars are created on the outside of the heart to create a transmural lesion. In other words, to achieve an ablated tissue through the thick muscle of the heart.

The present disclosure describes novel devices and methods for providing safe access to the heart, specifically transseptal and epicardial access, using radiofrequency energy. As will be explained in greater detail herein, the embodiments of the present disclosure improve and simplify the means for puncturing the heart, while preventing the chance of coring and providing enhanced manipulability by the user.

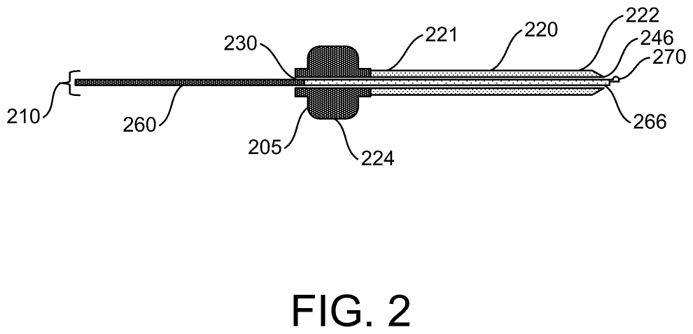

FIG. 2 is an illustration of a dilator 205 and an RF perforation device 210 according to an embodiment of the present disclosure. As shown, the dilator 205 includes a dilator body 220, a dilator hub 224, and a dilator lumen 230 extending longitudinally through the hub 224 and the dilator body 220. Additionally, the dilator body 220 has a proximal end portion 221 and an opposite distal end portion 222 terminating in a distal tip 246. The hub 224 is attached to the proximal end portion 221 of the dilator body 220. While the perforation device in FIG. 2 is described as a radiofrequency perforation device, in certain embodiments, the perforation device may be a mechanical perforation device.

As can be further seen from FIG. 2, the RF perforation device 210 includes a proximal portion 260 and a distal portion 266 extending from the proximal portion 260 and terminating in a distal functional tip 270 having a distal tip electrode (e.g., a tip electrode such as described above in connection with FIGS. 1A-1C). As will be appreciated, the length of the RF perforation device 210 is greater than the length of the dilator 205 so that part of the proximal portion 260 of the RF perforation device 210 extends proximally of the hub 224 when the distal portion 266, particularly the functional tip 270, extends distally of the dilator 205, thus allowing the proximal portion 260 to be manipulated by the user as needed.

In some embodiments, the proximal portion 260 of the RF perforation device 210 has an electrically insulated outer surface. As such, the proximal portion 260 can be handled directly by the user when the RF perforation device 210 is energized. In some embodiments, the proximal portion 260 is of a unitary construction formed entirely of an electrically insulative material. One exemplary class of materials for construction of the proximal portion can include various grades of polytetrafluoroethylene (PTFE), polyetheretherketone (PEEK), among others. In some embodiments, the proximal portion 260 can further include reinforcing elements, e.g., a polymeric braid or coil, to enhance the structural properties, e.g., stiffness, torque transfer capability, and the like. In some embodiments, the proximal portion is formed of a metal (e.g., a metal hypotube), and includes an outer electrically insulating layer.

In the illustrated embodiment, the distal portion 266 is electrically conductive and is capable of transferring radiofrequency energy supplied by an external RF generator to the functional tip 270 for subsequent delivery to the target tissue in a transseptal crossing or an epicardial ablation procedure, as described above. Any biocompatible electrically conductive material may be selected for construction of the distal portion 266. Exemplary materials may include stainless steel, nickel-titanium alloy, and the like. Further, for ease of illustration, the distal portion 266 is depicted in FIG. 2 as a single solid structure, although the construction of the distal portion 266 can vary to accommodate the particular structural requirements for the RF perforation device 210, as will be further explained below. For example, in some embodiments, the distal portion 266 can be constructed as a solid rod, a tube or a coil.

Additionally, in certain embodiments, the distal portion 266 can be constructed in multiple segments, e.g., a solid rod or hypotube in the regions nearest the proximal portion 260, and a coiled structure more distally to provide enhanced flexibility and torqueability. In some embodiments, the distal portion can have a composite construction, e.g., a solid or tubular core conductor surrounded by a wire coil. Additionally, as shown in the illustrated embodiment, the proximal and distal portions 260, 266 are substantially isodiametric, although this is not a strict requirement in all embodiments.

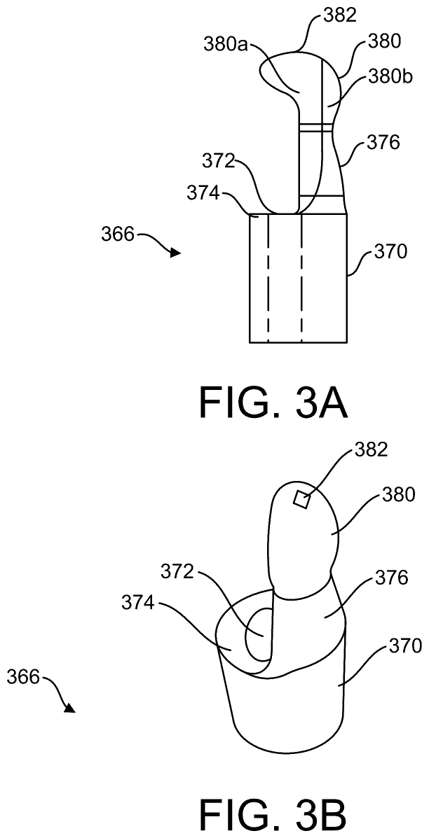

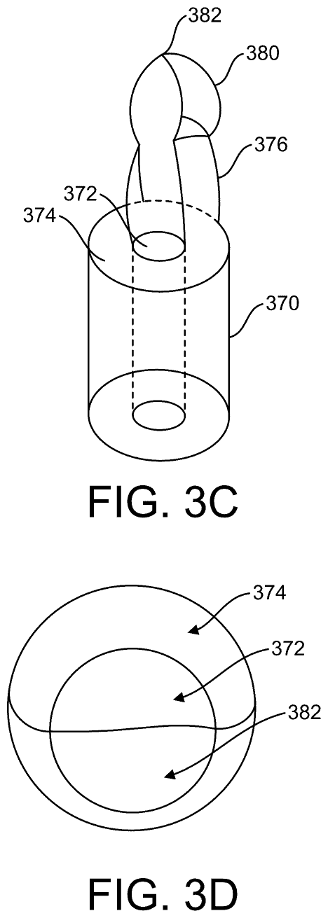

FIGS. 3A-3D are cross-sectional and schematic illustrations of a distal portion 366 of a RF perforation device for perforating a tissue, according to embodiments of the present disclosure. The RF perforation device of FIGS. 3A-3D may be substantially structurally and functionally identical to the RF perforation device of FIGS. 1A-1D and FIG. 2, except as described in connection with FIGS. 3A-3D herein. The RF perforation device includes an elongate member defining a lumen and extending from a proximal portion, as shown in FIG. 2, including a hub, to the distal portion 366. As shown, the distal portion 366 of the RF perforation device includes an open port housing 370 connected to a functional distal tip 380 by a tapered arm 376. The open port housing 370 includes an open port 372 having at least one aperture and an open port outer diameter. The functional distal tip 380 comprises a distal tip outer diameter, is defined by a distal face and includes a distal tip electrode 382 embedded to the distal end portion of the functional distal tip 380 used for the application of RF energy to a target tissue. As shown in FIGS. 3A-3C, the open port housing 370, which includes the open port 372, is proximate the functional distal tip 380. Additionally, the outer diameter of the functional distal tip 380 is less than the outer diameter of the open port 372. In some embodiments, as illustrated, the open port housing 370 further includes a proximal edge 374. In certain embodiments, the arm 376 which connects the open port housing 370 to the functional distal tip 380 is formed from a section of the proximal edge 374. In some embodiments, this configuration creates a functional distal tip 380 that protrudes next to and above the open port 372. In some embodiments, the distal tip 380 may include internal curvature and may be concave; while in other embodiments, the distal tip 380 may not include an internal curvature and may be flat.

In some embodiments, the distal face of the functional distal tip 380 is a half-rounded dome shape which is atraumatic to prevent accidental piercing of tissue. In some embodiments, the distal face of the functional distal tip 380 is blunt and includes no sharp edges. In certain embodiments, the shape of the distal face creates a functional distal tip 380 offset soft (i.e., not sharp) skive RF needle which reduces the risk of coring. In other embodiments, the distal face of the functional distal tip 380 may be a semi-spherical shape or any other geometric shape with a curved segment that lacks a sharp edge to prevent coring of tissue. In certain embodiments, the curve segment of the distal face allows for the safe advancement of the functional distal tip 380 through the perforation created by the distal tip electrode 382. In other embodiments, the curved segment of the distal face of the functional distal tip 380 may also reduce the risk of coring of tissue.

By creating the open port 372 with a protruding offset distal tip electrode 382, the RF perforation device may have a forward-facing opening while also being able to apply RF energy through the distal tip electrode 382 at a set distance away from the open port 372. In certain embodiments, this concentric configuration reduces the risk of coring while still enabling a 0.014″ or similar guidewire to be advanced through the elongate member, and more specifically through the open port 372 directly in line with the location of the perforation created by the distal tip electrode 382. In certain embodiments, the aperture at the open port 372 is connected to the lumen of the elongate member of the RF perforation device. In some embodiments, the open port 372 allows for the withdrawal of fluid, insertion of fluid through the aperture and/or allowing for passage of a guidewire through the aperture. In some embodiments, a user may introduce fluids, like saline or contrast, into the physiological system. In these embodiments, throughout the procedure, contrast may be injected to locate the functional distal tip 380 and the distal facing port may allow the contrast to exit the needle closer to the functional distal tip 380 and thus allow a user to locate the tip more easily. In other embodiments, a user may aspirate fluid to, for example, analyze the quality of the blood or be able to determine the location of the distal tip electrode 382. In certain embodiments, fluid may exit the open port 372 off-axis, i.e., fluid may exit at some angle to the longitudinal. For example, fluid may exit at an angle of between about 10 and about 90 degrees from the longitudinal axis. Additionally, in some embodiments, the creation of the forward-facing opening at the open port 372 allows for more accurate pressure readings and contrast injections at the distal portion 366. In certain embodiments, these pressure readings may include locating the position of the distal tip electrode 382 based on pressure reading differentials. In one embodiment, the pressure reading may determine whether the functional distal tip 380 has crossed the right atrium of the heart to the left atrium of the heart.

As discussed above and shown in FIGS. 3A-3C, the open port housing 370, which includes the open port 372, is proximate the functional distal tip 380. In some embodiments, while physically allowing access to the open port 372, the functional distal tip 380 may overshadow the open port lumen 372 and prevent the open port lumen 372 from directly being exposed to tissue. In certain embodiments, the height of the tapered arm 376 may be adjusted to be shorter or longer depending on the desired procedure and devices used. In some embodiments, as shown in FIG. 3A, if the tapered arm 376 is shorter in length, the functional distal tip 380 and/or the tapered arm 376 may be bent or curved towards the open port 372 to overshadow the open port lumen 372 and prevent the open port lumen 372 from being directly exposed to tissue. In certain embodiments, when the distal tip 380 is curved, the angle of approach of the distal tip 380 to the target tissue is between 30° and 60°. In other embodiments, the angle of approach of the functional distal tip 380 is between 35° and 55°. In still other embodiments, the angle of approach of the functional distal tip 380 is between 37° and 52°. The angle of approach is defined as the angle which will allow the open port 372 to be overshadowed by the distal tip 380 measured from the most distal portion of the open port 372 to the most distal portion of the distal tip 380. In some embodiments, if the functional distal tip 380 includes a curve towards the open port 372, the guidewire may still exit through the aperture of the open port 372 and is not obstructed by the functional distal tip 380. In other embodiments, if the tapered arm 376 is longer in height, the distal tip 380 may mask the open port 372 without the need for the distal tip 380 and/or the tapered arm 376 to be curved.

In one embodiment, the functional distal tip 380 may include two or more distal tip RF electrodes. In these embodiments, the distal tip 380 may include a deflection away from the center of the RF perforation device to allow for the guidewire to pass without being obstructed by the functional distal tip 380. In these embodiments, a longer arm 376 length may be used to allow for a better deflection away from the center of the RF perforation device to allow for the guidewire to pass the functional distal tip 380 and decrease the risk of the functional distal tip 380 from breaking off from the RF perforation device.

In some embodiments, as shown in FIG. 3A, an inner section 380a defining the inner portion of the functional distal tip 380, including the distal tip electrode 382, and the inner portion of the arm 376 may be electrically conductive and capable of transferring radiofrequency energy supplied by an external RF generator (not shown) to the distal tip electrode 382 for subsequent delivery to the target tissue in a transseptal crossing or an epicardial ablation procedure, as described above. Any biocompatible electrically conductive material may be selected for construction of the inner section 380a. Exemplary materials may include stainless steel, nickel-titanium alloy, and the like. In other embodiments, an outer section 380b defining the outer portion of the functional distal tip 380, including the curved segment of the functional distal tip 382, and the outer portion of the arm 376 is formed entirely of an electrically insulative material. One exemplary class of materials for construction of the outer section 380b can include various grades of polytetrafluoroethylene (PTFE), polyetheretherketone (PEEK), among others. In certain embodiments, the outer section 380b may further include reinforcing elements, e.g., a polymeric braid or coil, to enhance the structural properties, e.g., stiffness, torque transfer capability, and the like. In some embodiments, the proximal portion is formed of a metal (e.g., a metal hypotube), and includes an outer electrically insulating layer. In other embodiments the open port housing, including the open port 382 and the proximal edge 374 is formed entirely of an electrically insulative material. In some embodiments, the RF perforation device of the present invention may also be applied to devices in which a lumen with a port and an RF puncture feature at the distal end are beneficial. In some embodiments, the RF perforation device of the present invention may be applied to a catheter or a wire-based platform.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A radiofrequency perforation device for perforating a tissue, the device comprising:
an elongate member defining a lumen and extending from a proximal portion including a hub to a distal portion, the distal portion including an open port housing having at least one aperture and an open port outer diameter; and
a functional distal tip located at the distal portion of the elongate member having a distal face and including a distal tip electrode and a distal tip outer diameter;
wherein the distal portion further includes a tapered arm connecting the open port housing to the functional distal tip, wherein the open port housing is proximal to the functional distal tip and wherein the functional distal tip outer diameter is less than the open port housing outer diameter; and
wherein the distal face is a half-dome shape such that it reduces coring of the tissue.

2. The radiofrequency perforation device of claim 1, wherein the open port housing further includes a proximal edge that surrounds the aperture of the open port housing.

3. The radiofrequency perforation device of claim 2, wherein the tapered arm is formed from a section of the proximal edge.

4. The radiofrequency perforation device of claim 1, wherein the functional distal tip protrudes next to and overshadows the aperture without obstructing the aperture.

5. The radiofrequency perforation device of claim 1, wherein the distal face includes any geometric shape with a curved segment having no sharp edges.

6. The radiofrequency perforation device of claim 1, wherein the aperture is a forward-facing open port, and wherein the forward-facing open port facilitates the insertion of a guidewire from the proximal portion of the elongate member through to the forward-facing open port.

7. The radiofrequency perforation device of claim 6, wherein the functional distal tip is curved towards the open port to overshadow the open port without physically obstructing the open port.

8. The radiofrequency perforation device of claim 7, wherein the curved angle of the functional distal tip is between 35° and 55°.

9. The radiofrequency perforation device of claim 6, wherein the radiofrequency perforation device further includes a side port that is proximal to the open port.

10. The radiofrequency perforation device of claim 1, wherein the functional distal tip includes two or more distal tip RF electrodes.

11. The radiofrequency perforation device of claim 1, wherein a fluid exits the aperture, and wherein the fluid exits the aperture at an angle to the longitudinal axis.

12. An epicardial or transseptal crossing system for perforating a tissue, the system comprising:
a dilator having a dilator body defining a dilator lumen and a tapered distal tip;
an elongate member defining a lumen and extending from a proximal portion including a hub to a distal portion, the distal portion including an open port housing having at least one aperture and an open port outer diameter; and
a functional distal tip located at the distal portion of the elongate member having a distal face and including a distal tip electrode and a distal tip outer diameter;
wherein the distal portion further includes a tapered arm connecting the open port housing to the functional distal tip, wherein the open port housing is proximal to the functional distal tip and wherein the functional distal tip outer diameter is less than the open port housing outer diameter; and
wherein the distal face is a half-dome shape with no sharp edges, such that it reduces coring of the tissue.

13. The crossing system of claim 12, wherein the open port housing further includes a proximal edge that surrounds the aperture of the open port housing.

14. The radiofrequency perforation device of claim 13, wherein the tapered arm is formed from a section of the proximal edge.

15. The radiofrequency perforation device of claim 12, wherein the functional distal tip protrudes next to and overshadows the aperture without obstructing the aperture.

16. The radiofrequency perforation device of claim 12, wherein the aperture is a forward-facing open port.

17. The radiofrequency perforation device of claim 16, wherein the functional distal tip is curved towards the open port to overshadow the open port without physically obstructing the open port.

18. The radiofrequency perforation device of claim 12, wherein the distal portion further includes an inner section defining the inner portion of the functional distal tip, including the distal tip electrode, and the inner portion of the arm, and wherein the inner section is made of any biocompatible electrically conductive material and capable of transferring radiofrequency energy supplied by an external RF generator to the distal tip electrode and subsequent delivery to a target tissue.

19. The radiofrequency perforation device of claim 18, wherein the distal portion further includes an outer section defining the outer portion of the functional distal tip, including the curved segment of the functional distal tip, and the outer portion of the arm, and wherein the outer section is made of any electrically insulative material.

20. A method of epicardial or transseptal crossing, the method comprising:
providing an elongate member defining a lumen and extending from a proximal portion including a hub to a distal portion, the distal portion including an open port housing having at least one aperture and an open port outer diameter; and
advancing a functional distal tip located at the distal portion of the elongate member having a distal face and including a distal tip electrode and a distal tip outer diameter;
wherein the distal portion further includes a tapered arm connecting the open port housing to the functional distal tip, wherein the open port housing is proximal to the functional distal tip and wherein the functional distal tip outer diameter is less than the open port housing outer diameter; and
wherein the distal face is a half-dome shape such that it reduces coring of the tissue.
